(19) United States
(12) Patent Application Publication
(10) Pub. No.: US 2011/0052506 A1
(43) Pub. Date: Mar. 3, 2011

(54) Stable Aerosol Topical Foams Comprising a Hypochlorite Salt

(75) Inventors: Douglas Abel, Sudbury, MA (US); Ronald M. Gurge, Franklin, MA (US); Mark W. Trumbore, Westford, MA (US)

(73) Assignee: Collegium Pharmaceutical, Inc., Cumberland, RI (US)

(21) Appl. No.: 12/872,566

(22) Filed: Aug. 31, 2010

Related U.S. Application Data
(60) Provisional application No. 61/238,439, filed on Aug. 31, 2009.

(51) Int. Cl. A61K 9/12; A61P 17/00; A61P 31/00

(52) U.S. Cl. 424/45

(57) ABSTRACT

Described herein are compositions useful in the treatment of atopic dermatitis and other skin conditions, which compositions exhibit enhanced stability. The compositions contain a hypochlorite salt, useful for its antimicrobial properties, and are non-irritating when applied to the skin. The compositions also provide enhanced moisturizing properties. The compositions can be formulated into a topical aerosol foam with inert, non-flammable propellants, such as hydrofluoroalkanes, and may be used in cosmetics or pharmaceuticals.

### RELATED APPLICATION

This application claims benefit under 35 U.S.C. 119(e) of U.S. Provisional Application No. 61/238,439, filed on Aug. 31, 2009, the entire content of which is incorporated herein by reference.

### BACKGROUND

Atopic dermatitis (AD) is a disease characterized by dry, cracked, itchy, and inflamed skin, often presenting on greater than 10% of the body surface area. In accounts for 10-20% of all visits to a dermatologist and affects approximately 3% of the US population, most of whom are children. The condition is characterized by intense pruritus (itch) and a course marked by exacerbations and remissions. Higher transepidermal water loss (TEWL) has also been noted in dry skin atopic patients; TEWL is indicative of a disturbed barrier function, and it has been correlated to pruritus intensity in patients. Furthermore, the compromised skin barrier allows excessive water loss through the epidermal layer of the skin and the potential penetration of allergens.

Environmental factors, such as psychological stress, climate (e.g., low humidity caused by cold winters and central heating), and exposure to irritants and allergens determine the course of the disease. The defective barrier function associated with AD can also make atopic patients more prone to irritant contact dermatitis, since ordinary soaps and detergents often irritate the skin. Exposure to hard water, especially to calcium salts in domestic water, has been found to be associated with a higher prevalence of atopic eczema in primary-school children.

Another potential triggering factor for AD is the colonization of the skin with microorganisms, such as Staphylococcus aureus and Malassezia. S. aureus can release superantigenic exotoxins, which produce a massive release of cytokines Staphylococcus enterotoxin B also induces eczema when applied to uninvolved atopic and normal skin, while the severity of AD has been reported to correlate linearly with S. aureus counts.

A doctor has three main goals in designing a treatment regime for the patient: healing the skin and keeping it healthy, preventing flare ups, and treating symptoms when they do occur. Proper skin care and moisturizing ointments are the mainstays of topical treatment. Moisturizers which improve barrier function have been reported which reduce the prevalence of AD and can reduce the associated symptoms.

In addition to moisturizers, a variety of medications may be prescribed to help manage the condition. Topical steroids are the first-line treatment for atopic dermatitis flares because they are effective at reducing the inflammation caused by this disease. Immunomodulators (calcineurin inhibitors, such as tacrolimus and pimecrolimus) may also be prescribed. Immunomodulators change some of the functions of the immune system that cause atopic dermatitis without suppressing the whole immune system. Other immune-suppressing medications being investigated as a treatment for atopic dermatitis include: cyclosporine, interferon, methotrexate, and azothiaprine. Another mechanism of treatment includes the use of oral antihistamines, such as diphenhydramine or hydroxyzine. Oral antihistamines are used to treat itch associated with atopic dermatitis; however, they can cause sleepiness and may not help in all cases of atopic dermatitis.

For mild cases of atopic dermatitis, an over-the-counter formulation of coal tar is often used. Coal tar has long been a treatment for a variety of skin conditions. Shampoos and soaps containing coal tar can help with mild cases of atopic dermatitis. Coal tar tends to work better on thickened skin that is not scaly and the early symptoms of itching. However, coal tar can be irritating to already inflamed skin. Coal tar is used for mild cases of atopic dermatitis only.

For more severe flares of atopic dermatitis—for example if the rash covers a large part of the body or face—oral corticosteroids, such as prednisone, prednisolone, and medrol, may be used. Long-term use of oral steroids has numerous side effects, including weight gain, thinning of the bones, and suppression of the immune system; consequently, though they may clear atopic dermatitis well, the side effects are too risky to warrant using them as a first-line treatment. To avoid these side effects, but still benefit from the medication, oral steroids are often prescribed for a short course (e.g., five days) to calm the rash. Topical steroids can then be used on the remaining rash.

As discussed above, atopic dermatitis reduces the skin's natural defenses, making it easier for skin to become infected. If the skin becomes infected, antibiotics are often prescribed. Antibiotics, such as cefadroxil or cephalexin, are often prescribed at the first sign of infection.

Transient immersion of affected skin in a low concentration chlorine bleach bath (i.e., aqueous sodium hypochlorite) has been shown to decrease the microbial burden associated with atopic dermatitis, resulting in an improvement in symptoms. Skin and wound cleansers containing bleach have also been shown to reduce microbial skin contamination. To date, all bleach containing products for topical use are intended for transient skin contact, no leave-on products such as creams, lotions or topical foams intended for long term skin contact and containing bleach exist. Incorporation of bleach into leave-on products intended for long term skin contact allows for the potential to provide additional long-term treatment benefits such as improved moisturization and control of transepidermal water loss not possible with products intended for transient skin contact.

Hypochlorite salts are inherently unstable in aqueous solution. The decomposition rate of hypochlorite in water is dependent on concentration, temperature, and pH. High temperatures and acidic pHs greatly accelerate the rate of decomposition, as does the presence of metal ions. The normal shelf-life of bleach solutions is approximately six months at a pH of between 12 and 13.5. Additionally, due to the high pH and oxidative potential of bleach, bleach solutions are frequently irritating to the skin.

There exists a need for a stable, non-irritating topical formulation containing bleach suitable for the long-term application in the treatment of atopic dermatitis.

### SUMMARY OF THE INVENTION

In certain embodiments, the invention relates to a composition containing monovalent or divalent salts of hypochlorite, wherein the concentration of hypochlorite does not appreciably change with time. In certain embodiments, the composition is packaged into an aerosol can and pressurized with a hydrofluorocarbon propellant. In certain embodiments, when the can is actuated, a foam is dispensed. In certain embodiments, the dispensed foam is time- and temperature-stable, exhibits robust antimicrobial activity, moisturizes the skin, and is non-irritating.

### DETAILED DESCRIPTION OF THE INVENTION

In certain embodiments, the invention relates to a thickened aerosol foam composition containing a monovalent or divalent hypochlorite salt that is stable. In certain embodiments, when applied to the skin a composition of the invention reduces the number of skin-associated bacteria, yeasts, and fungi, improves skin moisture levels, and is non-irritating and non-drying. In certain embodiments, the invention relates to a composition that is suitable for the treatment of atopic dermatitis.

In one embodiment, the compositions do not contain volatile lower alcohols. In certain embodiments, the invention relates to a composition that does not comprise steroids.

In one embodiment, the compositions comprise an aerosol propellant. In one embodiment, the aerosol propellant is a hydrofluoroalkane (HFA) propellant.

In one embodiment, a composition produces a foam upon actuation of an aerosol container charged with the composition. In one embodiment, the foams are relatively stable against collapse. In one embodiment, the foams rub in quickly without a greasy, oily, or sticky residue. In one embodiment, the foam is moisturizing. In one embodiment, the foam is non-irritating. Application of the foam to the affected areas of a subject reduces the number of skin-associated bacteria, yeasts, and fungi, and improves skin moisture levels. In one embodiment, the composition rapidly and efficiently releases active ingredients.

### Propellants

There are a number of conceivable choices of propellants for a hypochlorite aerosol foam, including, but not limited to, CFCs, hydrocarbons, compressed gases, and hydrofluoroalkanes (HFAs). The Montreal Protocol has banned the use of CFCs (chlorofluorocarbons) due to their ability to deplete the ozone layer. Montreal Protocol on Substances that Deplete the Ozone Layer, United Nations Environmental Programme, 1987. Alternatively, hydrocarbon propellants demonstrate very low reactivity and good resistance to free-radical attack. However, hydrocarbon propellants are highly flammable and it would be undesirable and hazardous to combine these propellants with hypochlorite salts, strong oxidizers, in an aerosol foam system. The chemical classes of “oxidizer” and “flammable” are known to be incompatible. Finally, compressed inert gases, such as nitrogen and carbon dioxide, can be used as an aerosol propellant. While offering good chemical stability due to their non-reactivity, they are unable to deliver consistent product delivery throughout the life of the aerosol can due to their high vapor pressures. Another option is HFAs. These propellants are pharmaceutically acceptable, generally non-reactive, and ozone-friendly.

### Stabilization of Exemplary Compositions of the Invention

Remarkably, we have developed a stable aerosol foam formulation containing a hypochlorite salt and a fluorinated propellant. In one embodiment, the invention relates to the formation of a stable hypochlorite aerosol foam formulation, thus overcoming the expected and well-known stability issues associated with these chemicals.

In one embodiment, the compositions are formulated such that the chemical instability is reduced. For example, compositions were formulated with the addition of antioxidants to the concentrate. In one embodiment, the air in the container headspace may be replaced with an inert gas (argon). Compositions formulated in this way may exhibit improved stability in the presence of HFA propellants (e.g., HFA-134a and HFA-227).

### Moisturization and Irritation

Topical formulations of hypochlorite salts are known to be generally irritating and lack the ability to hydrate skin. These two negative attributes can lead to reduced patient compliance with its concomitant impact on therapeutic response. In one embodiment, the inventive aerosol foam formulations of hypochlorite salts are no more irritating than vehicle control. In one embodiment, the inventive aerosol foam formulations of hypochlorite salts demonstrate similar levels of erythema as intact untreated skin. In one embodiment, the inventive aerosol foam formulations have the ability to moisturize skin.

### Exemplary Compositions

In certain embodiments, the composition has a humectant concentration of about 5% to about 15% (by weight of the concentrate), a water concentration of about 60% to about 80% (by weight of the concentrate), a bleach concentration of about 0.0001% to about 1.5% (by weight of the concentrate), and a stabilizer concentration of about 0.5% to about 5.0% (by weight of the concentrate).

In one embodiment, the invention relates to a composition, comprising a concentrate and a propellant, wherein

the concentrate comprises
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate; and

the propellant is a hydrofluoroalkane propellant.

In one embodiment, the invention relates to a composition, consisting essentially of a concentrate and a propellant, wherein

the concentrate comprises
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate; and

the propellant is a hydrofluoroalkane propellant.

In one embodiment, the invention relates to a composition, consisting of a concentrate and a propellant, wherein

the concentrate comprises
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate; and

the propellant is a hydrofluoroalkane propellant.

In one embodiment, the invention relates to a composition, comprising a concentrate and a propellant, wherein

the concentrate comprises
an amount of a hypochlorite salt, wherein the amount of the hypochlorite salt is about 0.0001% to about 1.5% by weight of the concentrate;
an amount of a viscosity modifier, wherein the amount of the viscosity modifier is about 0.1% to about 6% by weight of the concentrate;
an amount of a surfactant, wherein the amount of the surfactant is about 0.01% to about 1% by weight of the concentrate;
an amount of water, wherein the amount of water is about 80% to about 99% by weight of the concentrate; and
an amount of a stabilizer, wherein the amount of the stabilizer is about 0.01% to about 1.0% by weight of the concentrate; and

the propellant is a hydrofluoroalkane propellant.

In one embodiment, the invention relates to any one of the above-mentioned compositions, wherein the hydrofluoroalkane propellant is 1,1,1,2-tetrafluoroethane, 1,1,1,2,3,3,3-heptafluoropropane, or a mixture thereof.

In one embodiment, the invention relates to any one of the above-mentioned compositions, wherein the hydrofluoroalkane propellant is 1,1,1,2-tetrafluoroethane.

In one embodiment, the invention relates to any one of the above-mentioned compositions, wherein the hypochlorite salt is present in an amount from about 0.0001% to about 1.5% by weight of the concentrate. In one embodiment, the invention relates to any one of the above-mentioned compositions, wherein the hypochlorite salt is present in an amount from about 0.001% to about 0.8% by weight of the concentrate. In one embodiment, the invention relates to any one of the above-mentioned compositions, wherein the hypochlorite salt is present in an amount from about 0.01% to about 0.5% by weight of the concentrate. In one embodiment, the invention relates to any one of the above-mentioned compositions, wherein the hypochlorite salt is present in an amount from about 0.1% to about 0.25% by weight of the concentrate. In one embodiment, the invention relates to any one of the above-mentioned compositions, wherein the hypochlorite salt is present in about 0.0001%, about 0.0005%, about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, or about 1.5% by weight of the concentrate.

In one embodiment, the invention relates to any one of the above-mentioned compositions, wherein the hypochlorite salt is sodium hypochlorite, potassium hypochlorite, calcium hypochlorite, magnesium hypochlorite, lithium hypochlorite, or copper(I) or copper(II)hypochlorite. In one embodiment, the invention relates to any one of the above-mentioned compositions, wherein the hypochlorite salt is sodium hypochlorite or calcium hypochlorite.

In one embodiment, the invention relates to any one of the above-mentioned compositions, wherein water is present in an amount from about 60% to about 80% by weight of the concentrate. In one embodiment, the invention relates to any one of the above-mentioned compositions, wherein water is present in an amount from about 65% to about 75% by weight of the concentrate. In one embodiment, the invention relates to any one of the above-mentioned compositions, wherein water is present in an amount from about 68% to about 72% by weight of the concentrate. In one embodiment, the invention relates to any one of the above-mentioned compositions, wherein water is present in about 60%, about 65%, about 70%, about 75%, or about 80% by weight of the concentrate. In one embodiment, the invention relates to any one of the above-mentioned compositions, wherein water is present in an amount from about 80% to about 99% by weight of the concentrate.

In one embodiment, the invention relates to any one of the above-mentioned compositions, wherein the stabilizer is selected from the group consisting of DL-alpha tocopheryl acetate, imidazolidinyl urea, diazolidinyl urea, phenoxyethanol, sodium methyl paraben, methylparaben, ethylparaben, propylparaben, potassium sorbate, sodium benzoate, sodium chloride, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, chlorobutanol, dichlorobenzyl alcohol, phenylethyl alcohol, benzyl alcohol, ascorbic acid, sodium bisulfite, butylated hydroxytoluene, butylated hydroxyanisole, α-tocopherol, sodium ascorbate, ascorbyl palmitate, propyl gallate, disodium EDTA, and mixtures thereof.

In one embodiment, the invention relates to any one of the above-mentioned compositions, wherein the stabilizer is DL-alpha tocopheryl acetate, methylparaben, propylparaben, disodium EDTA, or a mixture thereof. In one embodiment, the invention relates to any one of the above-mentioned compositions, wherein the stabilizer is sodium chloride.

In one embodiment, the invention relates to any one of the above-mentioned compositions, wherein the stabilizer is present in an amount from about 0.01% to about 1.0% by weight of the concentrate. In one embodiment, the invention relates to any one of the above-mentioned compositions, wherein the stabilizer is present in an amount from about 0.8% to about 4.0% by weight of the concentrate. In one embodiment, the invention relates to any one of the above-mentioned compositions, wherein the stabilizer is present in an amount from about 1.0% to about 3.0% by weight of the concentrate. In one embodiment, the invention relates to any one of the above-mentioned compositions, wherein the stabilizer is present in about 0.5%, about 0.8%, about 1.0%, about 1.5%, about 2.0%, about 2.5%, about 3.0%, about 3.5%, about 4.0%, about 4.5%, or about 5.0% by weight of the concentrate.

In one embodiment, the invention relates to any one of the above-mentioned compositions, wherein the humectant is selected from the group consisting of 2-ethylhexyl palmitate, sodium hyaluronate, glycerol, PPG-15 stearyl ether, lanolin alcohol, lanolin, cholesterol, petrolatum, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, lactic acid, butylene glycol, sodium PCA, carbowax 200, carbowax 400, carbowax 800, and mixtures thereof.

In one embodiment, the invention relates to the above-mentioned composition, wherein the humectant is 2-ethylhexyl palmitate, sodium hyaluronate, glycerol, petrolatum, dimethicone, propylene glycol, or a mixture thereof.

In one embodiment, the invention relates to any one of the above-mentioned compositions, wherein the humectant is present in an amount from about 18% to about 32% by weight of the concentrate. In one embodiment, the invention relates to any one of the above-mentioned compositions, wherein the humectant is present in an amount from about 20% to about 30% by weight of the concentrate. In one embodiment, the invention relates to any one of the above-mentioned compositions, wherein the humectant is present in an amount from about 22% to about 28% by weight of the concentrate. In one embodiment, the invention relates to any one of the above-mentioned compositions, wherein the humectant is present in about 15%, about 16%, about 17%, about 18%, about 19%, about 20%, about 22%, about 25%, about 28%, about 30%, about 32%, or about 35% by weight of the concentrate. In one embodiment, the humectant is present in an amount from about 5% to about 15% by weight of the concentrate.

In one embodiment, the invention relates to any one of the above-mentioned compositions, wherein the concentrate further comprises an emulsifier or a surfactant.

In one embodiment, the invention relates to any one of the above-mentioned compositions, wherein the emulsifier or the surfactant is selected from the group consisting of dicetyl phosphate, polyethylene glycol hexadecyl ether phosphate, polyoxyethylene monooctadecyl ether, polysorbate 20, polysorbate 40, polysorbate 60, polysorbate 80, steareth-10, sodium dodecyl sulfate, lauryl dimethyl amine oxide, cetyltrimethylammonium bromide (CTAB), polyoxyethylene sorbitan, octoxynol, N,N-dimethyldodecylamine-N-oxide, hexadecyltrimethylammonium bromide (HTAB), polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, methylbenzethonium chloride, behentrimonium methosulfate-cetearyl alcohol, emulsifying wax, polyoxyethylene oleyl ether, PEG-40 stearate, cetostearyl alcohol, ceteareth-12, ceteareth-20, ceteareth-30, ceteareth alcohol, glyceryl stearate, PEG-100 stearate, glyceryl stearate, PEG-100 stearate, steareth-2, steareth-20, stearamidopropyl dimethylamine, behentrimonium methosulfate, and mixtures thereof.

In one embodiment, the invention relates to any one of the above-mentioned compositions, wherein the emulsifier or the surfactant is dicetyl phosphate, polyethylene glycol hexadecyl ether phosphate, polyoxyethylene monooctadecyl ether, cetostearyl alcohol, or a mixture thereof.

In one embodiment, the invention relates to any one of the above-mentioned compositions, wherein the emulsifier or the surfactant is present in an amount from about 0.01% to about 1% by weight of the concentrate. In one embodiment, the invention relates to any one of the above-mentioned compositions, wherein the emulsifier or the surfactant is present in an amount from about 2% to about 10% by weight of the concentrate. In one embodiment, the invention relates to any one of the above-mentioned compositions, wherein the emulsifier or the surfactant is present in an amount from about 3% to about 9% by weight of the concentrate. In one embodiment, the invention relates to any one of the above-mentioned compositions, wherein the emulsifier or the surfactant is present in an amount from about 4% to about 8% by weight of the concentrate. In one embodiment, the invention relates to any one of the above-mentioned compositions, wherein the emulsifier or the surfactant is present in an amount from about 3% to about 7% by weight of the concentrate. In one embodiment, the invention relates to any one of the above-mentioned compositions, wherein the emulsifier or the surfactant is present in about 4%, about 5%, about 6%, about 7%, or about 8% by weight of the concentrate.

In one embodiment, the invention relates to any one of the above-mentioned compositions, wherein the concentrate further comprises a pH adjusting agent. In one embodiment, the invention relates to any one of the above-mentioned compositions, wherein the pH adjusting agent is sodium hydroxide. In various embodiments, the invention relates to any one of the above-mentioned compositions, wherein the pH adjusting agent is monobasic sodium phosphate, dibasic sodium phosphate, or a combination of monobasic sodium phosphate and dibasic sodium phosphate.

In one embodiment, the invention relates to any one of the above-mentioned compositions, wherein the concentrate further comprises a natural extract. In one embodiment, the natural extract is a fat or glycidic oil from Theobroma grandiflorum.

In one embodiment, the invention relates to any one of the above-mentioned compositions, wherein the composition is colorless. In one embodiment, the invention relates to any one of the above-mentioned compositions, wherein the composition is off-white.

In one embodiment, the invention relates to any one of the above-mentioned compositions, wherein the composition is low odor. In one embodiment, the invention relates to any one of the above-mentioned compositions, wherein the composition is fragrance-free.

In one embodiment, the invention relates to any one of the above-mentioned compositions, wherein the composition has a pH of from about 4.5 to about 7. In one embodiment, the invention relates to any one of the above-mentioned compositions, wherein the composition has a pH of from about 4.5 to about 6. In one embodiment, the invention relates to any one of the above-mentioned compositions, wherein the composition has a pH of about 4.5, about 5.0, about 5.5, or about 6.0.

In one embodiment, the invention relates to any one of the above-mentioned compositions, wherein the concentrate has a pH of from about 4.5 to about 7. In one embodiment, the invention relates to any one of the above-mentioned compositions, wherein the concentrate has a pH of from about 4.5 to about 6. In one embodiment, the invention relates to any one of the above-mentioned compositions, wherein the concentrate has a pH of about 4.5, about 5.0, about 5.5, or about 6.0.

In one embodiment, the invention relates to any one of the above-mentioned compositions, wherein the composition is in an aerosol container.

In one embodiment, the invention relates to any one of the above-mentioned compositions, wherein the composition is in an aerosol container, thereby forming a headspace of the aerosol container; and the headspace of the aerosol container is substantially free of oxygen.

In one embodiment, the invention relates to any one of the above-mentioned compositions, thereby forming a headspace of the aerosol container; and the headspace of the aerosol container consists essentially of argon.

In one embodiment, the invention relates to any one of the above-mentioned compositions, wherein when the aerosol container is actuated, the composition is expelled as a foam.

In one embodiment, the invention relates to any one of the above-mentioned compositions, wherein the composition is in an aerosol container. In one embodiment, the invention relates to any one of the above-mentioned compositions, wherein the composition is about 4% to about 50% propellant, by weight of the composition. In one embodiment, the invention relates to any one of the above-mentioned compositions, wherein the composition is about 5% to about 40% propellant, by weight of the composition. In one embodiment, the invention relates to any one of the above-mentioned compositions, wherein the composition is about 6% to about 30% propellant, by weight of the composition. In one embodiment, the invention relates to any one of the above-mentioned compositions, wherein the composition is about 6% to about 18% propellant, by weight of the composition. In one embodiment, the invention relates to any one of the above-mentioned compositions, wherein the composition is about 6%, about 7%, about 8%, about 9%, about 10%, about 11%, about 12%, about 13%, about 14%, about 15%, about 20%, about 25%, or about 30% propellant, by weight of the composition. In one embodiment, the invention relates to any one of the above-mentioned compositions, wherein the composition is about 12% propellant, by weight of the composition. In one embodiment, the invention relates to any one of the above-mentioned compositions, wherein about 6%, about 7%, about 8%, about 9%, about 10%, about 11%, about 12%, about 13%, about 14%, about 15%, about 20%, about 25%, or about 30% propellant, by weight of the composition, is required to deliver the concentrate as a stable foam.

In one embodiment, the invention relates to any one of the above-mentioned compositions, wherein the composition is in the form of a foam.

In one embodiment, the invention relates to any one of the above-mentioned compositions, wherein the composition produces a foam.

In one embodiment, the invention relates to any one of the above-mentioned compositions, wherein the foam is produced by actuation of an aerosol container comprising the composition.

In one embodiment, the invention relates to any one of the above-mentioned compositions, wherein the foam is non-irritating when applied to the skin of a subject.

In one embodiment, the invention relates to any one of the above-mentioned compositions, wherein the foam is moisturizing over a period of at least 8 hours when applied to the skin of a subject.

In one embodiment, the invention relates to any one of the above-mentioned compositions, wherein the composition does not comprise methanol, ethanol, propanols, or butanols.

In one embodiment, the invention relates to any one of the above-mentioned compositions, wherein the composition does not comprise methane, ethane, propane, butane, pentane, or hexane.

In one embodiment, the invention relates to any one of the above-mentioned compositions, wherein the composition is non-irritating when applied to the skin.

In one embodiment, the invention relates to any one of the above-mentioned compositions, wherein the composition is moisturizing when applied to the skin. In one embodiment, when applied to the skin, the composition is moisturizing over a period of at least 4, at least 6, at least 8, at least 10, or at least 12 hours. In one embodiment, when applied to the skin, the composition is moisturizing over a period of up to about 24 hours. In one embodiment, when applied to the skin, the composition is moisturizing over a period of up to about 48 hours. In one embodiment, when applied to the skin, the composition is moisturizing over a period of at least 8 hours.

In one embodiment, the invention relates to any one of the above-mentioned compositions, wherein the composition is non-sterile. In one embodiment, the invention relates to any one of the above-mentioned compositions, wherein the composition is sterile.

### Exemplary Methods of Use

In one embodiment, the present invention relates to a method of treating a condition of a subject in need thereof, comprising the steps of:

applying to an affected area of the subject an effective amount of a foam prepared from any one of the above-mentioned compositions.

In one embodiment, the present invention relates to any one of the above-mentioned methods, further comprising the step of:

expelling from an aerosol container any one of the above-mentioned compositions, thereby preparing a foam.

In one embodiment, the present invention relates to a method of treating a condition of a subject in need thereof, comprising the steps of:

applying to an affected area of the subject an effective amount of a foam prepared from any one of the above-mentioned compositions, thereby simultaneously treating and moisturizing the affected area.

In one embodiment, the present invention relates to a method of treating a condition of a subject in need thereof, comprising the steps of:

applying to an affected area of the subject an effective amount of a foam prepared from any one of the above-mentioned compositions, thereby simultaneously treating and hydrating the affected area.

In one embodiment, the present invention relates to the above-mentioned method, wherein the condition is atopic dermatitis, contact dermatitis, xerotic eczema, seborrhoeic dermatitis, psoriasis, dyshidrosis, discoid eczema, venous eczema, dermatitis herpetiformis, neurodermatitis, autoeczematization, herpes simplex I, other topical herpes or viral infections, common cold sores and fever blisters, ringworm, impetigo, or other viral, fungal, or bacterial infections of the skin. In one embodiment, the condition is a heavily contaminated or infected wound. In one embodiment, a heavily contaminated wound is understood by those of ordinary skill in the art to mean a wound that is heavily contaminated by micro-organisms, but not clinically infected. Such wounds are often characterized by a prolonged period of inflammation, as well as a delay in wound healing or repair. In one embodiment, heavily infected wounds are understood by those of ordinary skill in the art to mean wounds with a bioburden greater than 10⁵ micro-organisms per gram of tissue.

In one embodiment, the present invention relates to any one of the above-mentioned methods, wherein the condition is eczema.

In one embodiment, the present invention relates to any one of the above-mentioned methods, wherein the condition is atopic dermatitis. In one embodiment, the present invention relates to any one of the above-mentioned methods, wherein the condition is atopic dermatitis with more than about 10% body surface area (BSA) involvement. In one embodiment, the present invention relates to any one of the above-mentioned methods, wherein the condition is atopic dermatitis with up to about 80% body surface area (BSA) involvement.

In one embodiment, the present invention relates to any one of the above-mentioned methods, wherein the subject is human.

In one embodiment, the present invention relates to the above-mentioned method, wherein the affected area of the subject is the face, earlobes, neck, scalp, genitals, eyelids, palms, fingers, feet, exural (inner) surfaces of joints, extensor aspects of joints, or any combination thereof. In one embodiment, the present invention relates to the above-mentioned method, wherein the affected area of the subject is the face. In one embodiment, the present invention relates to the above-mentioned method, wherein the affected area of the subject is the exural surfaces of elbows or knees. In one embodiment, the present invention relates to the above-mentioned method, wherein the affected area of the subject is the extensor aspects of wrists, elbows, ankles, or knees.

In one embodiment, the present invention relates to any one of the above-mentioned methods, wherein the composition is applied once daily.

In one embodiment, the present invention relates to any one of the above-mentioned methods, wherein the composition is applied twice daily.

In one embodiment, the invention relates to a method of disinfecting an intact skin site prior to a surgical or invasive procedure.

### Exemplary Constituents of Compositions of the Present Invention

Exemplary identities of various constituents of the compositions of the present invention are described below.

### 1. Propellants

In one embodiment, the propellant is a HFA or a mixture of one or more hydrofluorocarbons. Suitable hydrofluorocarbons include 1,1,1,2-tetrafluoroethane (HFA-134a); 1,1,1,2,3,3,3-heptafluoropropane (HFA-227); and mixtures and admixtures of these and other HFAs that are currently approved or may become approved for medical use are suitable. Hydrocarbon as well as chlorofluorocarbon (CFC) propellants can also be used in the present invention.

### 2. Hypochlorite Salts

A variety of salts of hypochlorous acid (HOCl), both monovalent and divalent, may be present in a composition. These include sodium hypochlorite, potassium hypochlorite, calcium hypochlorite, magnesium hypochlorite, lithium hypochlorite, and copper(I) or copper(II)hypochlorite.

### 3. Other Active Agents

One or more additional active agents may be present in the composition. These include any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones.

### 3.1 Antibiotics

Representative antibiotics include, without limitation, octopirox, erythromycin, zinc, tetracyclin, triclosan, azelaic acid and its derivatives, phenoxy ethanol and phenoxy proponol, ethyl acetate, clindamycin and meclocycline; sebostats such as flavinoids; alpha and beta hydroxy acids; and bile salts, such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin.

### 3.2 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents.

### 3.3 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

In certain embodiments, steroids may be excluded from the compositions of the invention.

### 3.4 Anesthetics

Suitable anesthetics include the aminoacylanilide compounds such as lidocaine, prilocaine, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

### 3.5 Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

### 3.6 Keratolytic Agents

Suitable keratolytic agents include, but are not limited to, urea, salicylic acid, papain, sulfur, glycolic acid, pyruvic acid, resorcinol, N-acetylcysteine, retinoids such as retinoic acid and its derivatives (e.g., cis and trans, esters), alpha hydroxy acids, beta hydroxy acids, coal tar, and combinations thereof.

### 3.7 Other Agents

Suitable other agents include, but are not limited to, skin soothing agents, deodorant agents, antiperspirants, sun screening agents, sunless tanning agents, vitamins, hair conditioning agents, anti-irritants, anti-aging agents, and combinations thereof.

Examples of skin soothing agents include, but are not limited to, allantoin, aloe, avocado oil, green tea extract, hops extract, chamomile extract, colloidal oatmeal, calamine, cucumber extract, and combinations thereof.

Examples of vitamins include, but are not limited to, vitamins A, D, E, K, and combinations thereof.

Examples of sunscreens include, but are not limited to, p-aminobenzoic acid, Avobenzone, Cinoxate, Dioxybenzone, Homosalate, Menthyl anthranilate, Octocrylene, Octyl methoxycinnamate, Octyl salicylate, Oxybenzone, Padimate O, Phenylbenzimidazole sulfonic acid, Sulisobenzone, Titanium dioxide, Trolamine salicylate, Zinc oxide, 4-methylbenzylidene camphor, Methylene Bis-Benzotriazolyl Tetramethylbutylphenol, Bis-Ethylhexyloxyphenol Methoxyphenyl Triazine, Terephthalylidene Dicamphor Sulfonic Acid, Drometrizole Trisiloxane, Disodium Phenyl Dibenzimidazole Tetrasulfonate, Diethylamino Hydroxybenzoyl Hexyl Benzoate, Octyl Triazone, Diethylhexyl Butamido Triazone, Polysilicone-15, and combinations thereof.

### 4. Stabilizers

The composition may further include components adapted to improve the stability or effectiveness of the applied formulation. These include, but are not limited to, preservatives, buffers, antioxidants, and chelators.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols (such as α-tocopherol), DL-alpha tocopheryl acetate, sodium ascorbate/ascorbic acid, ascorbyl palmitate, propyl gallate, and chelating agents like ethylenediaminetetraacetic acid (EDTA, e.g., disodium EDTA), citric acid, and sodium citrate.

In addition, combinations or mixtures of these preservatives or anti-oxidants may also be used in the formulations of the present invention.

### 5. Surfactants and Emulsifiers

Many topical formulations contain chemical emulsions which use surface active ingredients (emulsifiers) to disperse dissimilar chemicals in a particular solvent system. For example, most lipid-like (oily or fatty) or lipophilic ingredients do not uniformly disperse in aqueous solvents unless they are first combined with emulsifiers which form microscopic aqueous soluble micelles that contain a lipid-soluble interior and an aqueous-soluble exterior, resulting in an oil-in-water emulsion. In order to be soluble in aqueous media, a molecule must be polar or charged so as to favorably interact with water molecules which are also polar. Similarly, to dissolve an aqueous-soluble polar or charged ingredient in a largely lipid or oil-based solvent, an emulsifier is typically used which forms stable micelles that contain the aqueous-soluble components in the micelle interior while the exterior of the micelle is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion micelle. Emulsion destabilization results in the aqueous and lipophilic ingredients separating into two layers, potentially destroying the commercial value of a topical product.

Surfactants suitable for use in the present invention may be ionic or non-ionic. These include, but are not limited to: dicetyl phosphate (1-hexadecanol, hydrogen phosphate), ceteth-10 phosphate (polyethylene glycol hexadecyl ether phosphate), polysorbates (Polysorbate 20, Polysorbate 40, Polysorbate 60, Polysorbate 80), steareth-10, sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, methylbenzethonium chloride, alkyl polyglucoside, e.g., Triton™ CG-110 (Dow Chemical Co.), ammonium lauroyl sarcosinate, e.g., Perlastan® AL-30 (Struktol, Stow, Ohio), sodium lauroyl sarcosinate, e.g., Perlastan® L-30 (Struktol, Stow, Ohio), and ammonium myristoyl sarcosinate, e.g., Perlastan® M-30 (Struktol, Stow, Ohio). Appropriate combinations or mixtures of such surfactants may also be used according to the present invention.

Many of these surfactants may also serve as emulsifiers in formulations of the present invention.

Other suitable emulsifiers for use in the formulations of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (C₁₆-C₁₈ alcohol), ceteareth-12, ceteareth-20, ceteareth-30, ceteareth alcohol, glyceryl stearate, PEG-100 stearate, glyceryl stearate and PEG-100 stearate, steareth-2, steareth-20, and polyoxyethylene monooctadecyl ether, or combinations/mixtures thereof, as well as cationic emulsifiers like stearamidopropyl dimethylamine and behentrimonium methosulfate, or combinations/mixtures thereof.

### 6. Vehicles

Suitable topical vehicles and vehicle components for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, formulations without methanol, ethanol, propanols, or butanols are desirable.

### 7. Moisturizers and Humectants

One of the most important aspects of topical products in general, and cosmetic products in particular, is the consumer's perception of the aesthetic qualities of a product. For example, while petrolatum is an excellent moisturizer and skin product, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers or humectants for use in the formulations of the present invention include, but are not limited to, lactic acid and other hydroxy acids and their salts, glycerol, propylene glycol, butylene glycol, sodium PCA, sodium hyaluronate, hyaluronic acid, Carbowax 200, Carbowax 400, and Carbowax 800.

Suitable humectants for use in the formulations of the present invention include, but are not limited to, 2-ethylhexyl palmitate (hexadecanoic acid, 2-ethylhexyl ester), glycerol, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, and dicaprylate/dicaprate.

In addition, appropriate combinations and mixtures of any of these moisturizing agents or humectants may be used in accordance with the present invention.

### 8. Viscosity Modifiers

Suitable viscosity adjusting agents (i.e., thickening and thinning agents) for use in the formulations of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

In one embodiment the viscosity modifier is hydrous sodium lithium magnesium silicate, e.g., Laponite® (Rockwood Additives Limited, Cheshire, UK).

In one embodiment the viscosity modifier is present in a composition of the invention in an amount from about 0.1% to about 6.0% by weight of the concentrate.

### 9. Additional Constituents

Additional constituents suitable for incorporation into the emulsions of the present invention include, but are not limited to: skin protectants, adsorbents, demulcents, moisturizers, buffering agents, sustained release materials, solubilizing agents, skin-penetration agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, and pH adjusters (e.g., citric acid, sodium hydroxide, and sodium phosphate).

Natural fats and oils, other than those listed above, may also be beneficial constituents of the inventive compositions. For example, fats and glyceridic oils from plants, such as Theobroma grandiflorum, may be added.

Suitable fragrances and colors may be used in the formulations of the present invention. Examples of fragrances and colors suitable for use in topical products are known in the art.

Often, one constituent of a composition may accomplish several functions. In one embodiment, the present invention relates to constituents that may act as a lubricant or a skin-penetrating agent. In one embodiment, the multi-functional constituent is socetyl stearate, isopropyl isostearate, isopropyl palmitate, or isopropyl myristate.

### 10. Purging Gases

In one embodiment, the air in the container charged with the composition is replaced by an inert gas. In certain embodiments, the inert gas is selected from the group consisting of argon, nitrogen, and mixtures thereof.

### Definitions

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

The invention now being generally described, it will be more readily understood by reference to the following examples, which are included merely for purposes of illustration of certain aspects and embodiments of the present invention, and are not intended to limit the invention.

### Examples

### Example 1

### Formulation

Four concentrates according to the invention were formulated as indicated in Table 1. Each column refers to two preparations; for example NB435-74/75 refers to NB435-77 and to NB435-75. Values shown are weight percentages of the total concentrate for each formulation. An HFA propellant in the form of HFA-134a was included in each formulation in an amount corresponding to 12.5% of the weight of the composition.

TABLE 1
Formulations of four concentrates

| | NB435- | NB435- | NB435- | NB489- |
|---|---|---|---|---|
| Ingredient (%) | 74/75 | 77/98 | 99/100 | 1/2 |
| Water | 96.3434 | 96.3434 | 96.3434 | 94.8434 |
| Hydrous Sodium Lithium | 3 | 3 | 3 | 4.5 |
| Magnesium Silicate¹ | | | | |
| Sodium Hypochlorite | 0.11 | 0.11 | 0.11 | 0.11 |
| Sodium Chloride | 0.0066 | 0.0066 | 0.0066 | 0.0066 |
| Sodium Phosphate, | 0.02 | 0.02 | 0.02 | 0.02 |
| Monobasic | | | | |
| Sodium Phosphate, | 0.02 | 0.02 | 0.02 | 0.02 |
| Dibasic | | | | |
| Alkyl | | | | 0.5 |
| Polyglucoside² | | | | |
| Ammonium Lauroyl | 0.5 | | | |
| Sarcosinate³ | | | | |
| Sodium Lauroyl | | 0.5 | | |
| Sarcosinate⁴ | | | | |
| Ammonium Myristoyl | | | 0.5 | |
| Sarcosinate⁵ | | | | |
| Total | 100.0000 | 100.0000 | 100.0000 | 100.0000 |

¹Laponite ®
²Triton ™ CG-110
³Perlastan ® AL-30
⁴Perlastan ® L-30
⁵Perlastan ® M-30

### Example 2

### Stability

The formulations described in Example 1 were packed in aerosol containers and evaluated for stability of the hypochlorite in each over a period of up to two and a half weeks at 25° C., 30° C., and 40° C. Aerosol containers were typical 1-inch aluminum can/aluminum valve aerosol configuration or 20 mm glass aerosol bottle/valve combination. Results are shown in Tables 2-4.

TABLE 2
Stability at 25° C.

| | 25° C. | | | |
|---|---|---|---|---|
| | NB435-75 | NB435-98 | NB435-100 | NB489-2 |
| Zero Time | 0.00940 | 0.01000 | 0.00911 | 0.01022 |
| 2.5 wk^{a} | 0.00213 | 0.00424 | 0.00400 | No Bleach |

^{a}typical 1-inch aluminum can/aluminum valve aerosol configuration

TABLE 3
Stability at 30° C.

| | 30° C. | | | |
|---|---|---|---|---|
| | NB435-74 | NB435-77 | NB435-99 | NB489-1 |
| Zero Time | 0.00958 | 0.01043 | 0.00924 | 0.01421 |
| 1 wk^{a} | 0.00339 (pH 9.4) | 0.00584 (pH 9.7) | 0.00536 (pH 9.8) | 0.00069 (pH 10.1) |
| 2 wk^{a} | Not Tested | 0.00458 (pH 10.0) | 0.00448 (pH 10.0) | Not Tested |
| 2 wk^{b} | 0.00171 (pH 9.2) | 0.00492 (pH 9.7) | 0.00455 (pH 10.0) | Not Tested |

^{a}typical 1-inch aluminum can/aluminum valve aerosol configuration
^{b}20 mm glass aerosol bottle/valve combination

TABLE 4
Stability at 40° C.

| | 40° C. | | | |
|---|---|---|---|---|
| | NB435-75 | NB435-98 | NB435-100 | NB489-2 |
| Zero Time | 0.00940 | 0.01000 | 0.00911 | 0.01022 |
| 1 wk^{a} | 0.00143 (pH 9.6) | 0.00421 (pH 9.6) | 0.00336 (pH 10.0) | No Bleach (pH 9.8) |
| 2 wk^{a} | Not Tested | 0.00129 (pH 9.7) | 0.00130 (pH 10.0) | Not Tested |
| 2 wk^{b} | Not Tested | 0.00280 (pH 9.1) | 0.00180 (pH 9.6) | Not Tested |

^{a}typical 1-inch aluminum can/aluminum valve aerosol configuration
^{b}20 mm glass aerosol bottle/valve combination

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A composition, comprising a concentrate and a propellant, wherein
the concentrate comprises
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate; and
the propellant is a hydrofluoroalkane propellant.

2. The composition of claim 1, wherein the composition is in an aerosol container.

3. The composition of claim 2, wherein when the aerosol container is actuated, the composition is expelled as a foam.

4. A method of treating a condition of a subject in need thereof, comprising
applying to an affected area of the subject an effective amount of a foam prepared from a composition of claim 1.

5. The method of claim 4, further comprising
expelling from an aerosol container a composition of claim 1, thereby preparing a foam.

6. The method of claim 4 or 5, wherein the subject is human.

7. The method of claim 6, wherein the affected area of the subject is selected from the group consisting of the face, earlobes, neck, scalp, genitals, eyelids, palms, fingers, feet, exural surfaces of joints, extensor aspects of joints, and any combination thereof

8. The method of claim 6, wherein the affected area of the subject is the face.

9. The method of claim 4 or 5, wherein the condition is selected from the group consisting of atopic dermatitis, contact dermatitis, xerotic eczema, seborrhoeic dermatitis, psoriasis, dyshidrosis, discoid eczema, venous eczema, dermatitis herpetiformis, neurodermatitis, autoeczematization, herpes simplex I, other topical herpes or viral infections, common cold sores and fever blisters, ringworm, impetigo, and other viral, fungal, or bacterial infections of the skin.

10. The method of claim 9, wherein the condition is atopic dermatitis.

11. The method of claim 9, wherein the subject is human.

12. The method of claim 4 or 5, wherein the composition is applied once daily or twice daily.

13. A composition, comprising a concentrate and a propellant, wherein
the concentrate comprises
an amount of a hypochlorite salt, wherein the amount of the hypochlorite salt is about 0.0001% to about 1.5% by weight of the concentrate;
an amount of a viscosity modifier, wherein the amount of the viscosity modifier is about 0.1% to about 6% by weight of the concentrate;
an amount of a surfactant, wherein the amount of the surfactant is about 0.01% to about 1% by weight of the concentrate;
an amount of water, wherein the amount of water is about 80% to about 99% by weight of the concentrate; and
an amount of a stabilizer, wherein the amount of the stabilizer is about 0.01% to about 1.0% by weight of the concentrate; and
the propellant is a hydrofluoroalkane propellant.

14. The composition of claim 13, wherein the composition is in an aerosol container.

15. The composition of claim 14, wherein when the aerosol container is actuated, the composition is expelled as a foam.

16. A method of treating a condition of a subject in need thereof, comprising
applying to an affected area of the subject an effective amount of a foam prepared from a composition of claim 13.

17. The method of claim 16, wherein the affected area of the subject is selected from the group consisting of the face, earlobes, neck, scalp, genitals, eyelids, palms, fingers, feet, exural surfaces of joints, extensor aspects of joints, and any combination thereof.

18. The method of claim 16, wherein the condition is selected from the group consisting of atopic dermatitis, contact dermatitis, xerotic eczema, seborrhoeic dermatitis, psoriasis, dyshidrosis, discoid eczema, venous eczema, dermatitis herpetiformis, neurodermatitis, autoeczematization, herpes simplex I, other topical herpes or viral infections, common cold sores and fever blisters, ringworm, impetigo, and other viral, fungal, or bacterial infections of the skin.

19. The method of claim 18, wherein the subject is human.

20. The method of claim 16, wherein the composition is applied once daily or twice daily.
